(19) United States
(12) Patent Application Publication
(10) Pub. No.: US 2014/0188074 A1
(43) Pub. Date: Jul. 3, 2014

(54) MEDICAL MARKING APPARATUS AND METHODS

(71) Applicants: Rachael Jacques, Plymouth, MN (US); Paul Howard Wessel, Delano, MN (US); Melanie Roberta Smerillo, Minnetrista, MN (US)

(72) Inventors: Rachael Jacques, Plymouth, MN (US); Paul Howard Wessel, Delano, MN (US); Melanie Roberta Smerillo, Minnetrista, MN (US)

(21) Appl. No.: 14/092,627

(22) Filed: Nov. 27, 2013

Related U.S. Application Data
(63) Continuation-in-part of application No. 13/486,639, filed on Jun. 1, 2012.
(60) Provisional application No. 61/492,226, filed on Jun. 1, 2011.
(60) Provisional application No. 61/731,424, filed on Nov. 29, 2012.
(60) Provisional application No. 61/820,002, filed on May 6, 2013.

(51) Int. Cl. A61M 5/42; G09B 23/28

(52) U.S. Cl. CPC A61M 5/427; G09B 23/285
USPC 604/506; 604/116; 434/262

(57) ABSTRACT

The present invention provides the materials and methods related to marking the injection site on a patient's skin. Specifically, the present invention provides the materials and methods for applying a medical marking tattoo containing at least one injecting targeting site, injecting a medical substance into the area of the injection target site, and marking the injecting targeting site using a tattoo activation element. The present invention also provides the materials and methods for a medical marking apparatus that can be used to perform the methods of the present invention to mark the injection site on a patient's skin. Other embodiments are also included herein.

This application is a continuation-in-part of U.S. patent application Ser. No. 13/486,639, filed Jun. 1, 2012, and further claims the benefit of U.S. Provisional Application Nos. 61/492,226, filed Jun. 1, 2011; 61/731,424, filed Nov. 29, 2012; and 61/820,002, filed May 6, 2013; the content of all of which is herein incorporated by reference in its entirety.

### TECHNICAL FIELD

This disclosure generally relates to the field of medical marking devices. This disclosure also relates to the application of a body treating material on the outer surface of the body where the injector is entirely supported during application or injection.

### BACKGROUND

According to the U.S. Department of Health and Human Services, Centers for Disease Control and Prevention, about 215,000 people aged 20 or younger have diabetes (Type I or Type 2). Between the years 2002-2005, 15,600 youths were diagnosed with Type 1 diabetes annually, and the rate of new cases for children aged 10 years or younger was 19.7 per 100,000 each year. Type I diabetes typically strikes children and young adults but the disease can occur at any age.

Type I diabetes (known as diabetes mellitus or juvenile-onset diabetes) develops when the body's immune system attacks and destroys the pancreatic beta cells. The beta cells are the only cells that produce the hormone insulin, which regulates glucose. Insulin helps the body transport the glucose contained in foods to cells throughout the body, where it is subsequently used for energy or stored. However, when beta cells are destroyed, the glucose remains in the blood, which leads to insulin resistance and serious organ damage.

Currently, there is no way to prevent Type I diabetes, and to survive, people must have insulin delivered by injection or pump. Diabetes management is complex and overwhelming, especially for parents (caregivers) and their newly diagnosed child. On a daily basis the caregivers must help their child count carbohydrates, test their blood, monitor exercise and inject or pump insulin into their bodies. Regular rotation of insulin sites is a standard recommendation for diabetes self-care. It is important that the injection site be regularly rotated to prevent lipohypertrophy and promote better absorption.

Failure to follow proper injection site rotation, for any reason, can have severe long-term consequences on the wellness of the diabetic adolescent and contribute to significant increases in future health care costs. Lipohypertrophy is the most common adverse consequence of inadequate insulin site rotation. Lipohypertrophy is a degenerative disorder of the subcutaneous tissue that can cause thickening of the tissue and lumps or dents in the affected areas. It can reduce absorption of insulin at the injection site by as much as 25%, leading to inferior glucose control. In some patients, lipohypertrophy results in higher total doses of insulin being injected into an individual, in order to achieve optimal glycemic control. Despite this severe health risk, a recent study concluded that many youths fail to adhere to an adequate site rotation plan (Survey of Insulin Site Rotation in Youth with Type 1 Diabetes Mellitus). Some of the reasons given for not adequately rotating the injection site involve comfort with the existing routine and concern with reaching unfamiliar injection sites.

Adequate injection site rotation is also important for controlling the symptoms of Multiple Sclerosis (MS), an autoimmune disease. MS is a disease in which the immune system attacks the brain and spinal cord. Scar tissue (sclerosis) or plaques form in the brain and/or spinal cord when, myelin, the protective sheath covering nerve fibers, are destroyed. Without this myelin, the electrical signals transmitted throughout the brain and spinal cord deteriorate, and the brain is unable to send and receive signals. The symptoms of MS are a result of the breakdown in this transmission.

The symptoms of MS vary from person-to-person and may include abnormal fatigue, tingling/pain, changes in vision, loss of balance and muscle coordination, depression/emotional changes, numbness, slurred speech, tremors, muscle spasticity, bladder and/or bowel problems, and in severe cases, partial or complete paralysis. The National Multiple Sclerosis Society estimates that there are approximately 300,000 to 400,000 people in the United States with MS, with approximately 200 people diagnosed each week. MS is two to three times as common in women as in men, with the first symptoms usually occurring between the ages of 20 and 40. No cure currently exists.

However, there are several drugs available that have been shown to slow the progression of MS and reduce the frequency and severity of MS attacks. Interferon beta-1a is used for the treatment of relapsing forms of MS and for treatment after an initial episode of MS. Interferon beta-1a is typically injected weekly into the muscle. Interferon beta-1b is also used for the treatment of relapsing forms of MS and is subcutaneously injected every other day. Due to the frequency of injection, most brand name interferon beta-1b drugs advise patients of the importance of injection site rotation and in some studies, injection site necrosis has been reported in 4% of patients in controlled trials (BETASERON® Safety Information 2011, http://www.betaseron.com/safety.jsp). Accordingly, there exists a need for tools to help patients and their caregivers implement adequate injection site rotation in chronic diseases such as Type I diabetes and MS. The present disclosure of medical marking devices and methods, as described below, addresses this limitation.

### SUMMARY

In general, failure to properly rotate the injection site can have severe long-term consequences on patient wellness and can significantly increase health care costs. Adequate injection site rotation is important for controlling the symptoms of diseases that require multiple injections during treatment, including Multiple Sclerosis (MS), diabetes, and other autoimmune diseases. A method for ensuring patient compliance with a protocol of adequate injection site rotation is therefore desirable.

The present invention relates generally to the materials and methods involved in marking an injection site on a patient's skin. Specifically, the present invention features the materials and methods for applying a medical marking tattoo containing at least one injection targeting site, injecting a medical substance into the area of the injection target site, and marking the injection targeting site using a tattoo activation element. The present invention also provides the materials and methods for a medical marking apparatus that can be used to perform the methods of the present invention to mark the injection site on a patient's skin.

In one aspect, the method of the present invention includes the application of a temporary medical marking tattoo that can contain between about 1 and about 200 different injection target sites. For example, the medical marking tattoo can contain about 1, 5, 10, 15, 20, 25, 30, 35, 40, 50, 60, 80, 100 or up to 200 injection targeting sites. In some embodiments, the medical marking can container a number of injection targeting sites that is a multiple of 7. In various embodiments the medical marking tattoo can include about 10 to 60 injection targeting sites. In some embodiments, the medical marking tattoo comprises a general design of an ornament, symbol, cartoon, popular fictional character, or a licensed trademark that is pleasing to children and young adults (e.g., FIG. 2). In some aspects, the medical marking tattoo can be a temporary skin tattoo made up of several layers of material interspersed with dye or ink-permeable layers, which can be visible on the patient's skin for a period of at least two weeks.

Once the medical marking tattoo has been applied, a patient, caregiver or other qualified professional can use an injection device to inject a medical substance at the injection target site delineated within the medical marking tattoo. In some aspects, a patient, caregiver or other qualified professional can inject a medical substance to treat diabetes, Multiple Sclerosis, other autoimmune diseases, cancer, growth disorders, or any other disease state that requires periodic injections for treatment. For example, a patient, caregiver or other qualified professional can inject any injectable medication. Injectable medications can include, but are not limited to, insulin, interferon beta-1a, interferon beta-1b, growth hormone, and the like. In some aspects, the medical substance injected by the injection device can be a nutritive substance designed to provide sustenance to a patient.

In some aspects, the medical marking apparatus can consist of an injection device bonded (e.g., thermally or mechanically) to a tattoo activation element, wherein the tattoo activation element is used to mark the injection target site before, after, or simultaneously with the injection of the medical substance (e.g., FIG. 3). In some aspects, the medical marking apparatus can consist only of a tattoo activation element, and the injection device can be used separately (e.g., FIG. 4). The tattoo activation element can mark the area around the injection target site using a needle and syringe, a piston pump or a plunger and barrel, all of which can be used to deliver medical ink or dye to the area of the injecting targeting site. In this way, the tattoo activation element provides visual confirmation of where the injection occurred and can ensure that the injection target site is properly rotated for the next dose of medication. In some aspects, the medical ink or dye used to mark the injection targeting site can be contained within a protective cap on the tattoo activation element (e.g., FIG. 7). The tattoo activation element can also be used to deliver medical dye or ink that can be magnetically coupled to a medical marking tattoo. In some aspects, the medical marking apparatus can further comprise an infusion pump, an infusion catheter, an insertable needle assembly, or an adhesive patch (e.g., FIG. 8). Once a medical substance has been injected and the injection site is marked, the method of the present invention can be repeated, or a new medical marking tattoo can be applied to another area of the patient's skin and the process can begin again.

In an embodiment, the invention includes an injection site marking device. The injection site marking device can include a marker and a removable cap. The marker can include a syringe channel and an ink retaining media. The ink retaining media can include an ink. The ink retaining media can be disposed on an end of the marker. Ink can be disposed in the ink retaining media. The syringe channel can be configured to fit a syringe head. The syringe channel can extend over the length of the marker. The removable cap can be configured to engage the marker.

### BRIEF DESCRIPTION OF THE DRAWINGS

FIG. 1 is a flow chart illustrating an exemplary embodiment of a method for applying a medical marking tattoo on the body and injecting a medical substance at an injection target site indicated on the tattoo.

FIG. 2 is a schematic illustration of an exemplary embodiment of a medical marking tattoo according to the present disclosure.

FIG. 3 is a schematic illustration of an exemplary medical marking apparatus according to the present disclosure.

FIG. 4 is a schematic illustration of an exemplary embodiment of a tattoo activation element according to the present disclosure.

FIG. 5 is an alternative medical marking apparatus according to the present disclosure.

FIG. 6 is a flow chart illustrating an exemplary alternative embodiment of a method for a medical marking apparatus tattoo.

FIG. 7 is a schematic illustration of an exemplary alternative medical marking apparatus according to the present disclosure.

FIG. 7a is a schematic illustration of a protective cap of a medical marking apparatus.

FIG. 7b is a schematic illustration of a protective cap of a medical marking apparatus.

FIG. 8 is a schematic illustration of an exemplary alternative medical marking apparatus according to the present disclosure.

FIG. 9 is a schematic illustration of an exemplary embodiment of the medical marking apparatus used on a patient's body.

FIG. 10 is a schematic view of an injection site marking device in conjunction with a syringe in accordance with various embodiments herein.

FIG. 11 is a schematic view of an injection site marking device in conjunction with a syringe in accordance with various embodiments herein.

FIG. 12 is a cross-sectional schematic view of an injection site marking device in conjunction with a syringe in accordance with various embodiments herein.

FIG. 13 is a schematic view of a marker in accordance with various embodiments herein.

FIG. 14 is a schematic view of a removable cap in accordance with various embodiments herein.

FIG. 15 is a schematic view of ink retaining media in accordance with various embodiments herein.

FIG. 16 is a schematic view medical marking tattoo including day and time indicia in accordance with various embodiments herein.

FIG. 17 is an exploded schematic view of elements of a kit in accordance with various embodiments herein.

FIG. 18 is a schematic view of elements of a kit in accordance with various embodiments herein.

FIG. 19 is a schematic view of an injection site marking device in accordance with various embodiments herein.

FIG. 20 is a schematic view of an injection site marking device in accordance with various embodiments herein.

FIG. 21 is a schematic view of an injection site marking device in accordance with various embodiments herein.

FIG. 22 is a schematic view of an injection site marking device in accordance with various embodiments herein.

FIG. 23 is a schematic view of an infusion set marking device in accordance with various embodiments herein.

### DETAILED DESCRIPTION OF THE INVENTION

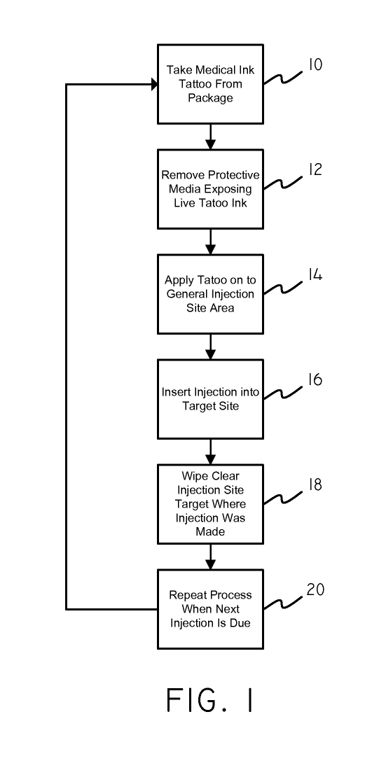

FIG. 1 illustrates an exemplary embodiment of a method of applying a medical ink tattoo and injecting a medical substance at the injection site. This method (100) includes removing the medical marking ink tattoo from the manufacturer's packaging (10); removing the protective media and exposing live tattoo ink (12); applying the tattoo to the general injection site area (14); inserting the injection device at the injection target site (16); wiping the injection target site with an antiseptic after injection of the medical substance (18); and then repeating the process when medically necessary (20).

The term ‘medical marking tattoo’ is used to describe any temporary surface treatment applied to the skin of an individual. Thus, ‘medical marking tattoo’ technology may include several layers of FDA approved materials interspersed with dye or ink-permeable and dye or ink-impermeable layers, so the individual's skin is exposed to the dye or ink and a tattoo will form from the layers permeable to the dye or ink. The individual may cut out and remove a pattern from the layers that transfers onto the skin when dye or ink is applied on one side.

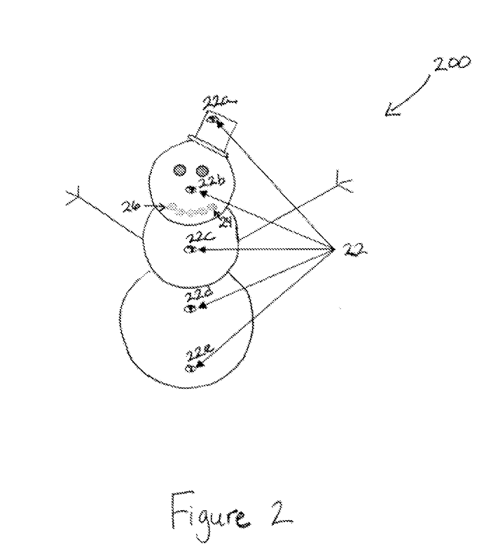

Referring to FIG. 2, exemplary medical marking tattoo (200) includes easily identifiable target sites (22a, 22b, 22c, 22d, and 22e). The medical marking tattoo is a temporary skin tattoo in the general design of an ornament, symbol, cartoon, popular fictional character, or a licensed trademark that is pleasing to children and young adults. Exemplary medical marking tattoo (200) is configured into the shape of a snowman and has five injection target sites (22a, 22b, 22c, 22d, and 22e). In one embodiment, two additional injection target sites may be included so that a medical marking tattoo may be used for up to one week. In another embodiment, a medical marking tattoo may contain enough injection target sites to last two weeks. In some embodiments, the medical marking tattoo can include a glow-in-the-dark material such as a glow-in-the-dark dye or ink. In some embodiments, the medical marking tattoo can include a material that fluoresces under UV light.

In some embodiments, the medical marking tattoo can include a surface topology. By way of example, in some embodiments, the surface of the medical marking tattoo can be characterized by a plurality of raised portions. In some embodiments, the raised portions can be high enough to be perceptible by a user with their fingertips. In some embodiments, the plurality of raised portions can be a plurality of raised dots, similar to braille. In some embodiments, the plurality of raised portions can be raised lines or ridges. In some embodiments, the plurality of raised portions can define shapes such as circles, squares, rectangles, or the like.

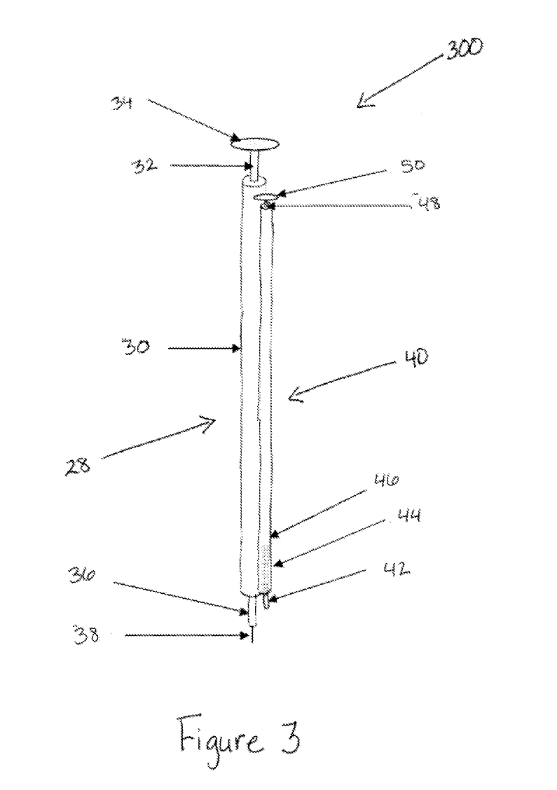

Referring to FIG. 3, an exemplary medical marking apparatus (300) includes a syringe (28) containing medicine (e.g., insulin) and is bonded to a tattoo activation element (40). In one embodiment, the syringe is a piston pump which includes a plunger (32) that extends throughout the length of the barrel (30) of the syringe in order to expel liquid or gas from the fluid reservoir. Located at one end (proximal) of the plunger is a flat surface perpendicular to a plunger, called a thumb or finger pad (34). Pressure is applied to the thumb or finger pad in order to expel liquid or gas from a syringe. A tensile force may also be applied to the thumb or finger pad in order to draw in liquid or air. At the other end (distal) of a syringe, there may be a needle or other injection device (38) which punctures the skin at the injection target site and delivers a medical substance (e.g., insulin). A needle can range from 5 to 330 mm in length, and from 25 to 33-gauge in thickness, or any other reasonable size depending on the individual patient. Finally, the barrel of a syringe may taper to a needle by way of a fitting (36). Such a fitting may be of a luer lok® design, a slip fit design, a catheter tip design, an eccentric tip design, or any other design suited to the individual patient.

FIG. 3 also refers to a tattoo activation element (40), which is coaxially aligned with the axis of tan injection device using a bonding process. The bonding process may be thermal or mechanical. A tattoo activation element functions similarly to a syringe. It may include a plunger (48) that extends throughout the barrel (46) of a tattoo activation element in order to expel liquid or gas from the fluid reservoir. Located at one of (proximal) end of the plunger is a flat surface perpendicular to a plunger called a thumb or finger pad (50). Pressure is applied to a thumb or finger pad in order to expel liquid or gas from the tattoo activation element. A tensile force may also be applied to the thumb or finger pad 50 in order to draw in liquid or air. At the other end (distal) of the tattoo activation element, there is a needle (42) through which medical ink (44) is delivered to the skin at, or in close proximity to, the injection target site where a medical substance was injected or applied (e.g., insulin). A needle can range from 5 to 330 mm in length, and from 25 to 33-gauge in thickness, although the size may vary according to the individual patient. Alternatively, the tattoo activation element can be contained within a syringe.

The medical ink or dye (44) is preferably FDA-approved. In one embodiment, medical ink or dye is of a different pigmentation than the medical marking tattoo (200) of FIG. 2. In another embodiment, the medical ink or dye, after application, is in the general design of an ornament, symbol, cartoon, popular fictional character, or a licensed trademark that is pleasing to children and young adults. In another embodiment, an antiseptic agent is delivered prior to, or after, the medical ink or dye is applied so that the injection target sites are sterilized. In another embodiment, medical ink or dye is magnetically coupled to the medical marking tattoo (200) of FIG. 2. After the skin is marked with medical ink or dye, the patient and his or her caregiver has visual confirmation of where the injection occurred and can ensure that the injection target site is properly rotated for the next dose of medication. At this time, the method of applying a medical marking tattoo may be repeated.

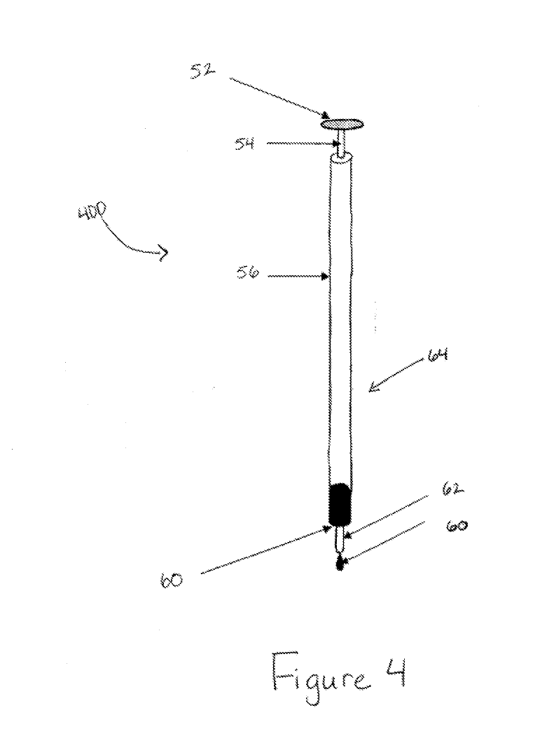

Referring to FIG. 4, an exemplary medical marking apparatus (400) includes a tattoo activation element (64) that expels medical ink or dye (60), without being coupled to a syringe that injects medication. A tattoo activation element may also include a plunger (54) that extends throughout the barrel (56) of the tattoo activation element in order to expel liquid or gas. Located at one of (proximal) end of the plunger is a flat surface, perpendicular to the plunger, called a thumb or finger pad (52). Pressure is applied to the thumb or finger pad in order to expel liquid or gas from tattoo activation element. A tensile force may also be applied to the thumb or finger pad in order to draw in liquid or air. At the other end (distal) of tattoo activation element there is a needle (62), through which medical ink or dye is delivered to the skin at, or in close proximity to, the injection target site where a medical substance was injected or applied (e.g., insulin). A needle can range from 5 to 330 mm in length, and from 25 to 33-gauge in thickness, although the size may vary depending on the individual patient.

The medical ink or dye is preferably FDA-approved. In one embodiment, medical ink or dye is of a different pigmentation than the medical marking tattoo (200) of FIG. 2. In another embodiment, medical ink or dye, after application, is in the general design of an ornament, symbol, cartoon, popular fictional character, or a licensed trademark that is pleasing to children and young adults. In another embodiment, an antiseptic agent is delivered prior to, or after, the medical ink or dye is applied so that the injection target sites are sterilized. In another embodiment, medical ink or dye is magnetically coupled to the medical marking tattoo (200) of FIG. 2. After the skin is marked with medical ink or dye, the patient and his or her caregiver has visual confirmation of where the injection occurred and can ensure that the injection target site is properly rotated for the next dose of medication. At this time, the method of applying a medical marking tattoo may be repeated.

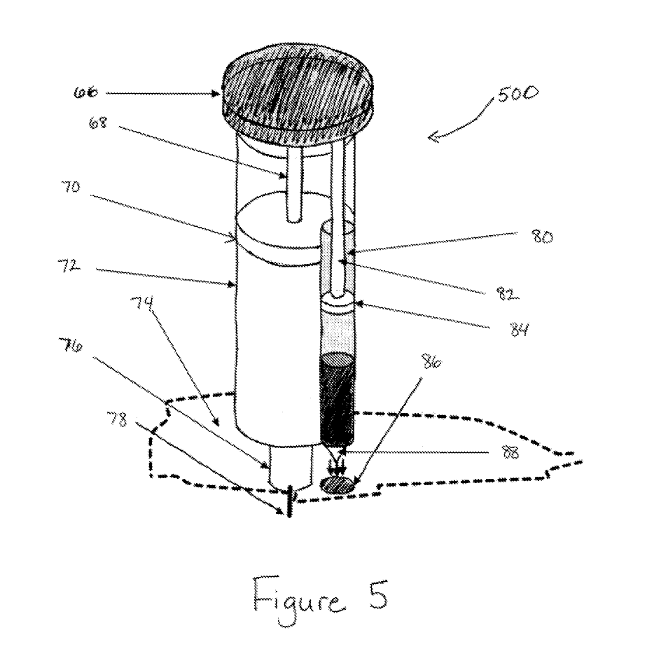

Referring to FIG. 5, a medical marking apparatus (500) is depicted that includes a syringe (70) operably connected to a tattoo activation element (84) by a primary plunger (68).

The syringe is a piston pump that includes a primary plunger that extends throughout the barrel (72) of the syringe in order to expel liquid or gas. Located at one of end (proximal) of the primary plunger is a flat surface perpendicular to primary plunger called a thumb or finger pad. Pressure is applied to the thumb or finger pad in order to expel liquid or gas from the syringe. A tensile force may also be applied to the thumb or finger pad in order to draw in liquid or air. At the other end (distal) of the syringe, 70 there may be a needle or other injection device (78) which punctures the skin at the injection target site (74) and delivers a medical substance (e.g., insulin). The needle can range from 5 to 330 mm in length, and from 25 to 33-gauge in thickness, or any other reasonable size depending on the individual patient. Finally, the barrel (72) of the syringe may taper to a needle by way of a fitting (76). The fitting may be of a luer lok® design, a slip fit design, a catheter tip design, an eccentric tip design, or any other design suited to the individual patient.

The tattoo activation element is coaxially aligned with the axis of an injection device by way of a bonding process. The bonding process may be thermal or mechanical. A tattoo activation element may have a secondary plunger (80 and 82) connected to the thumb or finger pad of a syringe, such that medical ink or dye (86) is not distributed from a needle (88) onto the target injection site until the primary plunger has traveled the length of the barrel.

The medical ink or dye is preferably FDA-approved. In one embodiment, the medical ink or dye is of a different pigmentation than the medical marking tattoo (200) of FIG. 2. In another embodiment, the medical ink or dye, after application, is in the general design of an ornament, symbol, cartoon, popular fictional character, or a licensed trademark that is pleasing to children and young adults. In another embodiment, an antiseptic agent is delivered prior to, or after, the medical ink or dye 86 so the injection target sites are sterilized. In another embodiment, the medical ink or dye is magnetically coupled to the medical marking tattoo (200) of FIG. 2. After the skin is marked with medical ink or dye, the patient and his or her caregiver has visual confirmation of where the injection occurred and can ensure that the injection target site is properly rotated for the next dose of medication. At this time, the method of applying a medical marking tattoo may be repeated.

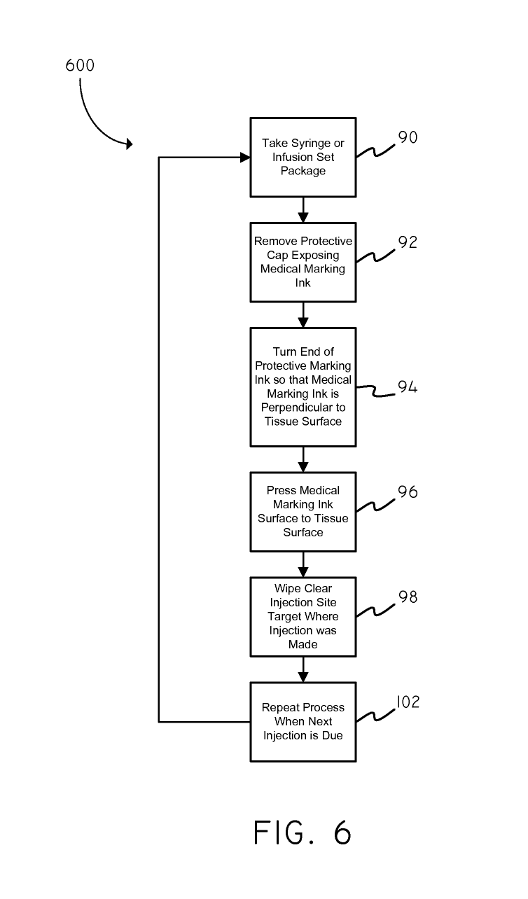

FIG. 6 illustrates an alternative embodiment of a method (600) for applying a medical marking at an injection site. This method includes procuring the medical marking kit from the manufacturer (90); removing the protective cap from the syringe (92); positioning the cap opening perpendicular to the surface of the skin (94); pressing the cap downward (96); wiping the injection target site with an antiseptic (98); and repeating the process at the next injection target site as medically needed (102).

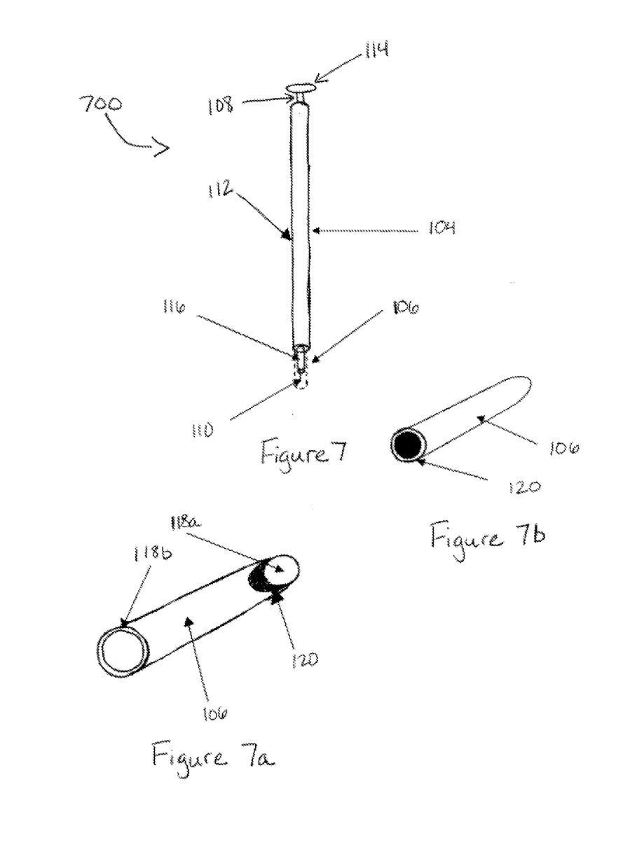

Referring to FIG. 7, a medical marking apparatus (700) may include a syringe (104) with protective cap (106). A syringe may be a piston pump which includes a plunger (108) that extends throughout the barrel (112) of the syringe in order to expel liquid or gas. Located at one of end (proximal) of the plunger is a flat surface perpendicular to the plunger called a thumb or finger pad (114). Pressure is applied to the thumb or finger pad in order to expel liquid or gas from the syringe. A tensile force may also be applied to the thumb or finger pad in order to draw in liquid or air. At the other end (distal) of the syringe there may be a needle (110) which punctures the skin at the injection target site and delivers a medical substance (e.g., insulin). A needle can range from 5 to 330 mm in length, and from 25 to 33-gauge in thickness, or any other reasonable size depending on the individual patient. Finally, the barrel of the syringe may taper to a needle by way of a fitting (116). The fitting may be of a luer lok® design, a slip fit design, a catheter tip design, an eccentric tip design, or any other design suited to the individual patient.

Referring to FIG. 7a, the protective cap of a medical marking apparatus (700) may include medical ink or dye (120) encompassed within the closed end (118a) of the protective cap. After a medical substance has been delivered to the injection target site with the needle, the open end (118b) of the protective cap is positioned perpendicular to the skin's surface and force is applied. The force displaces the medical ink or dye to the opening of the protective cap, as shown in FIG. 7b, and medical ink or dye is applied to the surface of the skin. After the skin is marked with medical ink or dye, the patient and his or her caregiver has visual confirmation of where the injection occurred and can ensure that the injection target site is properly rotated for the next dose of medication. At this time, use of medical marking apparatus may be repeated.

The medical ink or dye is preferably FDA-approved. In one embodiment, the medical ink or dye is of a different pigmentation than the medical marking tattoo (200) of FIG. 2. In another embodiment, the medical ink or dye, after application, is in the general design of an ornament, symbol, cartoon, popular fictional character, or a licensed trademark that is pleasing to children and young adults. In another embodiment, an antiseptic agent is delivered prior to, or after, medical ink or dye so that the injection target sites are sterilized. In another embodiment, the medical ink or dye is magnetically coupled to the medical marking tattoo (200) of FIG. 2.

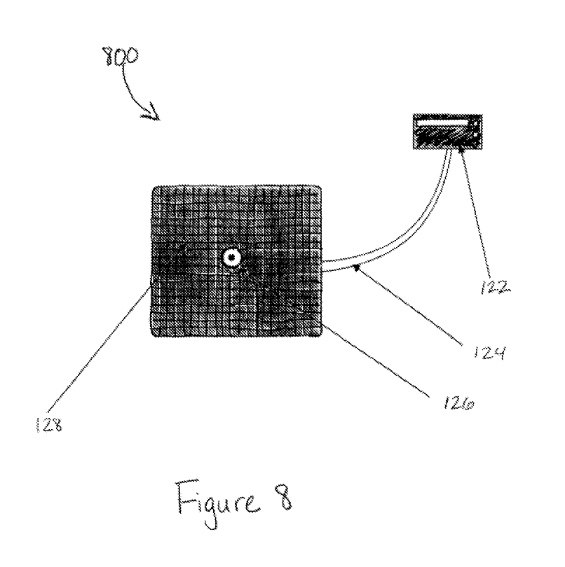

Referring to FIG. 8, a medical marking apparatus (800) may include an infusion pump (122), an infusion catheter (124), an insertable needle assembly (126), of an adhesive patch (128). An adhesive patch may contain medical ink or dye. The medical marking apparatus can be adapted so medical ink or dye is deposited onto the surface of the skin after the adhesive patch has been positioned. Alternatively, an adhesive patch of a medical marking apparatus can also contain an antiseptic agent working simultaneously with the medical ink or dye.

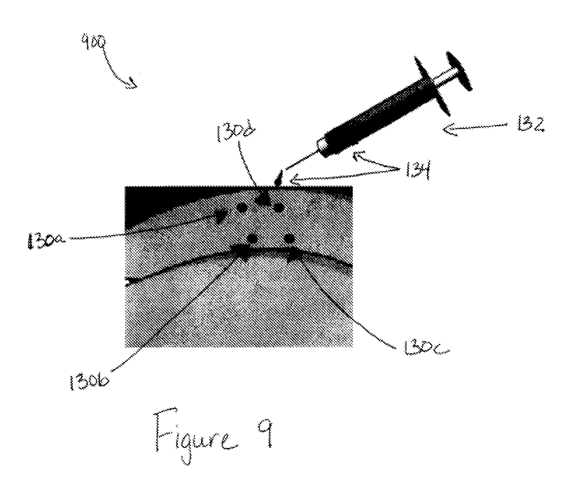

Referring to FIG. 9, the general injection site (900) may include easily identifiable target sites (130a, 130b, 130c, and 130d). A tattoo activation element (132) may contain a medical ink or dye (134) which is a different pigmentation than the tattoo left on a target site After the appropriate medical injection has been made into target sites, the patient is left with medical ink or dye on the surface of his or her skin for up to two weeks. After the skin is marked with medical ink or dye, the patient and his or her caregiver has visual confirmation of where the injection occurred and can ensure that the injection target site is properly rotated for the next dose of medication.

In an embodiment, the invention includes an injection site marking device. The injection site marking device can include a marker and a removable cap. The marker can include a syringe channel and an ink retaining media. The ink retaining media can include an ink. The ink retaining media can be disposed on an end of the marker. Ink can be disposed in the ink retaining media. The syringe channel can be configured to fit a syringe head. The syringe channel can extend over the length of the marker. The removable cap can be configured to engage the marker. Contacting the ink retaining media with patient's skin can result in deposition of a pattern of ink. In some embodiments, the pattern of ink can contain at least one injection target site. In some embodiments, the pattern of ink can include various indicia such as described herein.

The injection site marking device can serve various purposes. By way of example, the injection site marking device can be used in conjunction with methods of identifying if an injection has been administered since the injection will result in contact between the marking device and the patient's skin leaving a visible mark. Similarly, the injection site marking device can be used in conjunction with methods of aiding injection site rotation since the mark left on the patient's skin after an injection will serve as a visual identifier to a person administering a later shot. In addition, the injection site marking device can serve as an aid to drug identification, day, or time of administration because the color of ink provided by the marking device can be chosen from a selection of different colors and can serve to identify a particular drug, day or time of administration based on the particular color.

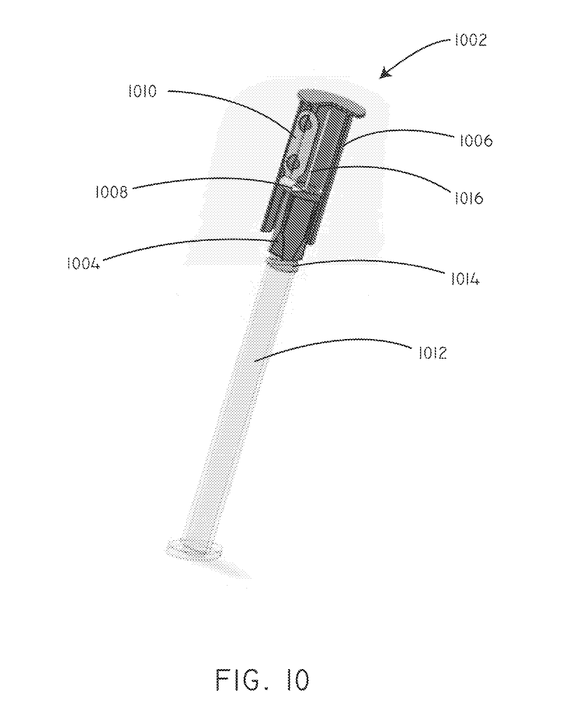

Referring now to FIG. 10, the injection site marking device 1002 includes a marker 1004 and a removable cap 1006. The injection site marking device 1002 can include a foil liner 1008. The foil liner 1008 can serve to keep ink fresh until the removable cap 1006 is removed. The foil liner 1008 can include a foil liner tab 1010. The foil liner tab 1010 can be used to assist in removing the foil liner 1008 when the removable cap 1006 is removed. The injection site marking device 1002 can engage syringe 1012. The syringe 1012 can include syringe head 1014. The syringe 1012 can also include syringe needle 1016.

In some embodiments, the color of the marker and/or the removable cap can vary (beyond the color of the ink as mentioned above). For example, in some embodiments, injection site marking devices of different colors can be used to correspond to different types of medications. This can be beneficial to visually distinguish different types of medications from one another. In some embodiments, injection site marking devices can be provided as a kit including multiple injection site marking devices in various quantities with different colors. In some embodiments, indicia can be disposed on the marker and/or the removable cap corresponding to different types of medications. The indicia can include distinguishing colors, designs, surface characteristics, raised portions, printing, and the like. In some embodiments, the color of the marker and/or the removable cap can match the color of the ink within the marker.

Referring now to FIG. 11, the marker 1004 is shown with the cap removed with the syringe ready for use. The marker 1004 includes an ink retaining media 1118. The ink retaining media 1118 can define an aperture (or central lumen) through which the needle of the syringe passes. The injection site marking device 1002 engage syringe 1012.

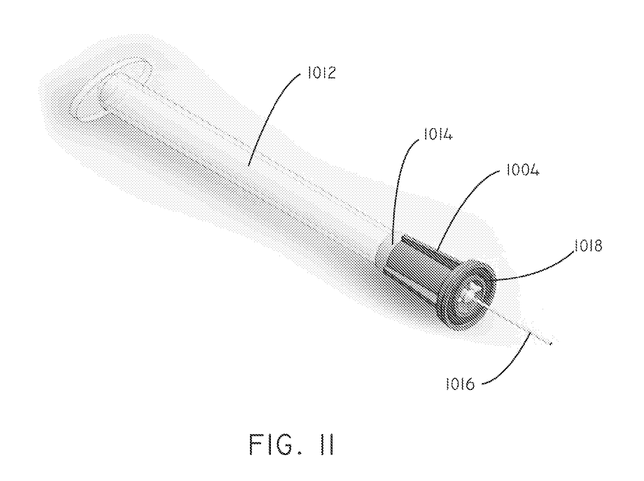

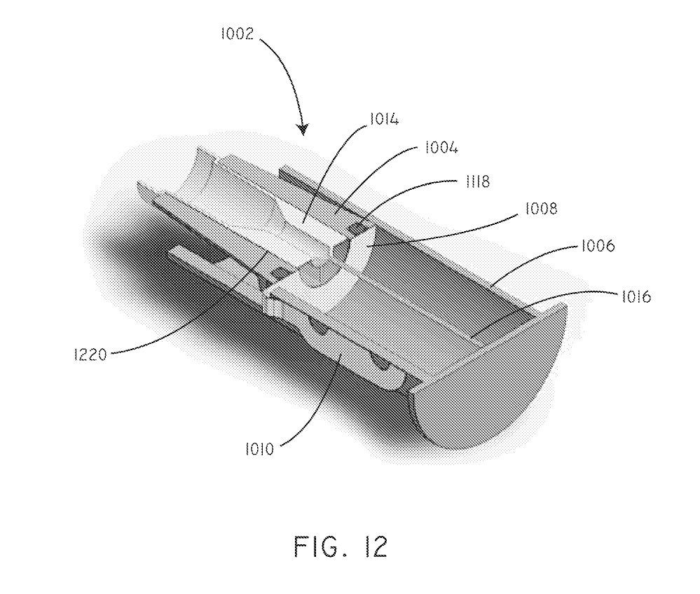

Referring now to FIG. 12, a sectional view of the injection site marking device 1002 is shown. The injection site marking device 1002 includes a marker 1004 and a removable cap 1006. The marker 1004 includes a syringe channel 1220 and an ink retaining media 1118. The injection site marking device 1002 can include foil liner 1008. The foil liner 1008 can include foil liner tab 1010. The foil liner tab 1010 can connect the foil liner 1008 and the removable cap 1006. The ink retaining media 1118 can define an aperture. The foil liner 1008 can be disposed over the ink retaining media 1118. The foil liner 1008 can be removed from over the ink retaining media 1118 when the removable cap is removed.

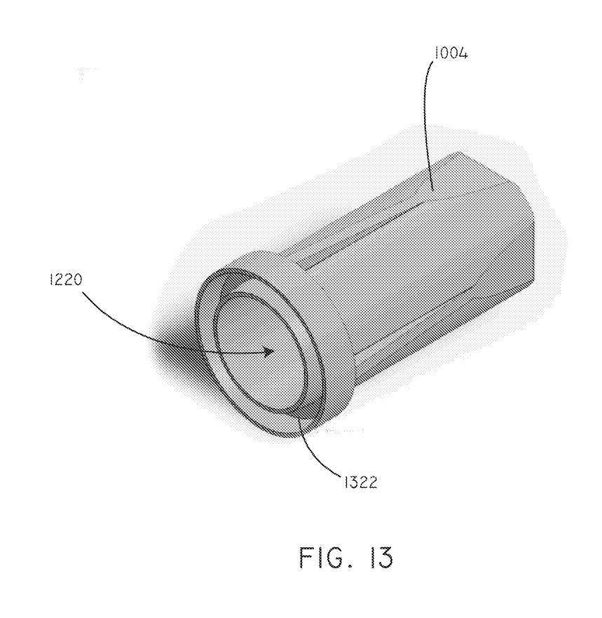

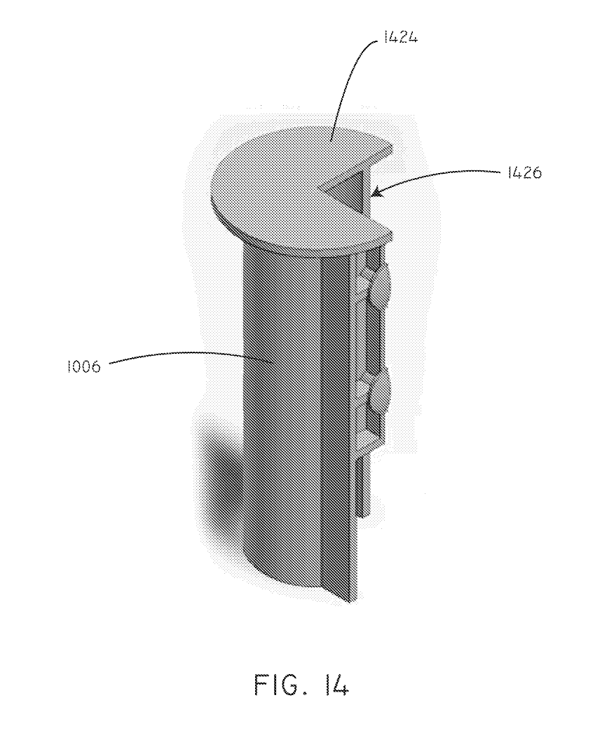

Referring now to FIG. 13, the marker 1004 includes a syringe channel 1220. The syringe channel 1220 can extend over the length of the marker 1004. The marker 1004 can also include a recessed portion 1322 into which the ink retaining media 1118 can fit. However, in other embodiments, the ink retaining media 1118 is not disposed within a recessed portion. Referring now to FIG. 14, the removable cap 1006 can include cap head 1424. The removable cap 1006 can also include cut-away segment 1426. The cut-away segment 1426 can allow viewing of the syringe needle when a syringe is positioned within the injection site marking device.

Referring now to FIG. 15, a schematic view of one example of ink retaining media 1118 is shown. The ink retaining media 1118 can be a porous medium that holds and then releases ink upon contact with a surface. The ink retaining media 1118 can define an aperture 1528 (or central lumen) through which the needle of the syringe passes. The ink retaining media 1118 and the aperture 1528 can be of various dimensions.

It will be appreciated that the syringe channel can be configured to fit over a syringe head. The syringe channel can have various dimensions in order to fit many different sizes of syringes. The syringe channel can extend over the length of the marker.

The ink retaining media can be disposed on an end of the marker. Ink can be disposed in the ink retaining media. Many different types of ink can be included. By way of example, inks can include, but are not limited to permanent inks, temporary inks, UV fluorescing inks, glow-in-the-dark inks, and the like. The term “inks” as used herein shall include dyes and other forms of colorants. The removable cap can be configured to engage the marker. The removable cap can be configured to fit on the marker with a snap-fit type mechanism. In some embodiments, the removable cap is configured to fit on the marker with a breakage mechanism that allows a user to determine if the cap was previously removed from the marker.

In some embodiments, the foil liner can include a metal foil. In some embodiments, the foil liner can include a polymer film. In still other embodiments, the foil liner can include a composite material. The foil liner can include foil liner tab. The ink retaining media can be ring shaped. The ink retaining media can define an aperture or central lumen. In some embodiments, the aperture can be at least about 1 mm, 2 mm, 3 mm, 4mm, or 5 mm in diameter. The removable cap can include cap head. In some embodiments the cap head can have a larger diameter than the rest of the removable cap. The removable cap can include a cut-away segment in some embodiments.

The marker can be formed of a polymer, a cellulosic material, a composite, or the like. The removable cap can be formed of a polymer, a cellulosic material, a composite, or the like. The syringe channel can include threads to engage corresponding threads on a syringe. In some embodiments, the syringe channel can include a snap fitting to engage the syringe. In some embodiments, the syringe channel can include a compression fitting to engage the syringe. The body of the marker itself and/or the cap can be colored to match the color of the ink that the ink retaining media carries.

In various embodiments, a method of preventing infection is included herein. The method can include applying a medical marking tattoo containing at least one injection target site to a patient's skin where an injection is to be made. The method can further include injecting a medical substance into said injection target site contained within said medical marking tattoo using an injection device, wherein the space between the injection target site and previous injection target sites is indicated by the medical marking tattoo. In some embodiments, the space between the injection target site and previous injection target sites as indicated by the medical marking tattoo is between about 0.25 inches and 2.0 inches. In some embodiments, the space between the injection target site and previous injection target sites as indicated by the medical marking tattoo is at least about 0.25 inches. In some embodiments, the space between the injection target site and previous injection target sites as indicated by the medical marking tattoo is at least about 0.5 inch. In some embodiments, the space between the injection target site and previous injection target sites as indicated by the medical marking tattoo is at least about 1.0 inch. In some embodiments, the method can also include removing the medical marking tattoo from the patient's skin. In some embodiments, removing the medical marking tattoo can include applying an antiseptic wipe to the medical marking tattoo surface. In some embodiments, removing the medical marking tattoo can include applying an aqueous solution to the medical marking tattoo surface. In some embodiments, removing the medical marking tattoo can include removing a portion of the tattoo corresponding to the current injection site. Removing a portion of the tattoo can be performed in conjunction with each injection performed.

In various embodiments, a method of preventing scar formation is included herein. The method can include applying a medical marking tattoo containing at least one injection target site to a patient's skin where an injection is to be made. The method can further include injecting a medical substance into said injection target site contained within said medical marking tattoo using an injection device, wherein the space between the injection target site and previous injection target sites is indicated by the medical marking tattoo. In some embodiments, the space between the injection target site and previous injection target sites as indicated by the medical marking tattoo is between about 0.25 inches and 2.0 inches. In some embodiments, the space between the injection target site and previous injection target sites as indicated by the medical marking tattoo is at least about 0.25 inches. In some embodiments, the space between the injection target site and previous injection target sites as indicated by the medical marking tattoo is at least about 0.5 inch. In some embodiments, the space between the injection target site and previous injection target sites as indicated by the medical marking tattoo is at least about 1.0 inch. In some embodiments, the method can also include removing the medical marking tattoo from the patient's skin. In some embodiments, removing the medical marking tattoo can include applying an antiseptic wipe to the medical marking tattoo surface. In some embodiments, removing the medical marking tattoo can include applying an aqueous solution to the medical marking tattoo surface. In some embodiments, removing the medical marking tattoo can include removing a portion of the tattoo corresponding to the current injection site. Removing a portion of the tattoo can be performed in conjunction with each injection performed.

In various embodiments, a method of injection site identification and rotation is included herein. The method can include applying a medical marking tattoo containing at least one injection target site to a patient's skin where an injection is to be made. The method can further include injecting a medical substance into said injection target site contained within said medical marking tattoo using an injection device, wherein the space between the injection target site and previous injection target sites is indicated by the medical marking tattoo. In some embodiments, the space between the injection target site and previous injection target sites as indicated by the medical marking tattoo is between about 0.25 inches and 2.0 inches. In some embodiments, the space between the injection target site and previous injection target sites as indicated by the medical marking tattoo is at least about 0.25 inches. In some embodiments, the space between the injection target site and previous injection target sites as indicated by the medical marking tattoo is at least about 0.5 inch. In some embodiments, the space between the injection target site and previous injection target sites as indicated by the medical marking tattoo is at least about 1.0 inch. In some embodiments, the method can also include removing the medical marking tattoo from the patient's skin. In some embodiments, removing the medical marking tattoo can include applying an antiseptic wipe to the medical marking tattoo surface. In some embodiments, removing the medical marking tattoo can include applying an aqueous solution to the medical marking tattoo surface. In some embodiments, removing the medical marking tattoo can include removing a portion of the tattoo corresponding to the current injection site. Removing a portion of the tattoo can be performed in conjunction with each injection performed.

In various embodiments, a method of removing a medical tattoo is included herein. The method can include applying an aqueous solution to the medical marking tattoo surface. In some embodiments, removing the medical marking tattoo can include removing a portion of the tattoo corresponding to the current injection site. Removing a portion of the tattoo can be performed in conjunction with each injection performed.

In various embodiments, a method of applying a medical tattoo is included herein. The method can include holding a carrier sheet with the tattoo disposed thereon against the skin of a patient. The method can further include contacting the carrier sheet with an aqueous solution of cause the tattoo to stick to the patient's skin and peel off the carrier sheet.

In some embodiments, a method of educating a newly diagnosed patient on proper injection site management is included herein. The method can include providing a newly diagnosed patient with a medical marking tattoo. The medical marking tattoo can include one such as described herein. In some embodiments, the method can include providing the newly diagnosed patient with an injection kit, such as described below. In some embodiments, the method can include selecting patients that are newly diagnosed with a condition requiring therapy that includes injection of a therapeutic agent. In some embodiments, the method can include selecting patients that have not received training regarding injection site rotation. In some embodiments, the method can include holding a carrier sheet with the tattoo disposed thereon against the skin of a patient. The method can further include contacting the carrier sheet with an aqueous solution to cause the tattoo to stick to the patient's skin and peel off the carrier sheet.

In some embodiments, a method of educating a previously diagnosed patient on proper injection site management is included herein. The method can include providing a previously diagnosed patient with a medical marking tattoo. The medical marking tattoo can include one such as described herein. In some embodiments, the method can include providing the previously diagnosed patient with an injection kit, such as described below. In some embodiments, the method can include selecting patients that are previously diagnosed with a condition requiring therapy that includes injection of a therapeutic agent, but newly exhibiting negative consequences and/or symptoms from improper injection site management. Negative consequences and/or symptoms from improper injection site management can include, but are not limited to, lipohypertrophy, subcutaneous infiltrate of a therapeutic agent, infection, excessive bleeding, cellulitus, and the like. In some embodiments, the method can include selecting patients that have not received training regarding injection site rotation. In some embodiments, the method can include holding a carrier sheet with the tattoo disposed thereon against the skin of a patient. The method can further include contacting the carrier sheet with an aqueous solution to cause the tattoo to stick to the patient's skin and peel off the carrier sheet.

In some embodiments, a method of educating a care giver on proper injection site management is included herein. The method can include the care giver with a medical marking tattoo. The medical marking tattoo can include one such as described herein. In some embodiments, the method can include providing the care giver with an injection kit, such as described below. In some embodiments, the method can include selecting care givers who have not previously received training on proper injection site rotation. In some embodiments, the method can include selecting care givers based on the type of patients they treat. In some embodiments, the method can include holding a carrier sheet with the tattoo disposed thereon against the skin of a patient. The method can further include contacting the carrier sheet with an aqueous solution to cause the tattoo to stick to the patient's skin and peel off the carrier sheet.

In some embodiments, a method of temporary reinforcement of proper injection site rotation is included herein. The method can include providing a patient with a medical marking tattoo. The medical marking tattoo can include one or more such as those described herein. In some embodiments, the method can include providing the patient with an injection kit, such as described below. In some embodiments, the method can include selecting patients that are receiving therapy that includes injection of a therapeutic agent. In some embodiments, the method can include selecting patients that have not received training regarding injection site rotation. In some embodiments, the method can include holding a carrier sheet with the tattoo disposed thereon against the skin of a patient. The method can further include contacting the carrier sheet with an aqueous solution to cause the tattoo to stick to the patient's skin and peel off the carrier sheet.

In some embodiments, a method of communicating information amongst caregivers as to the location of an injection is included. In some embodiments, the method can include a care giver holding a carrier sheet with the tattoo disposed thereon against the skin of a patient. Wherein the tattoo includes multiple injection target sites, distinguished from one another by indicia on the tattoo that includes information selected from the group consisting of day information and time information. In some embodiments, day information can include information regarding particular days. In some embodiments, time information can include information regarding parts of a day, including but not limited to, morning, afternoon, and night. For example, in some embodiments, the tattoo can include injection target sites arranged in columns and rows, wherein the columns identify different days and the rows identify different times or time periods within a day. In some embodiments, the space between adjacent injection target sites as indicated by the medical marking tattoo is at least about 0.5 inches.

In some embodiments, a method of tracking reactions to an injection is included. The method can include applying a medical marking tattoo containing at least one injection target site or a pattern of ink containing at least one injection target site to a patient's skin where an injection is to be made; injecting a medical substance into said injection target site contained within said medical marking tattoo using an injection device; and evaluating the injection target site for evidence of a reaction.

In some embodiments, a method of preventing, tracking, and/or identifying medication error is included herein. The method can include applying a medical marking tattoo containing at least one injection target site to a patient's skin where an injection is to be made; and injecting a medical substance into said injection target site contained within said medical marking tattoo using an injection device.

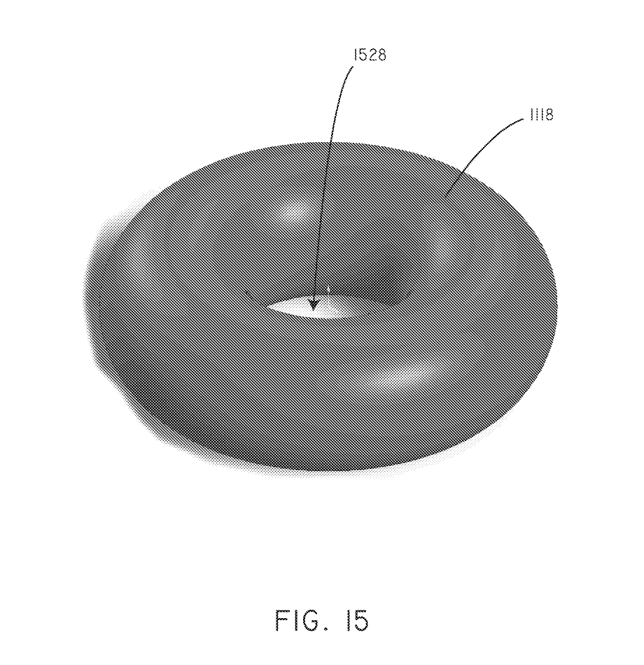

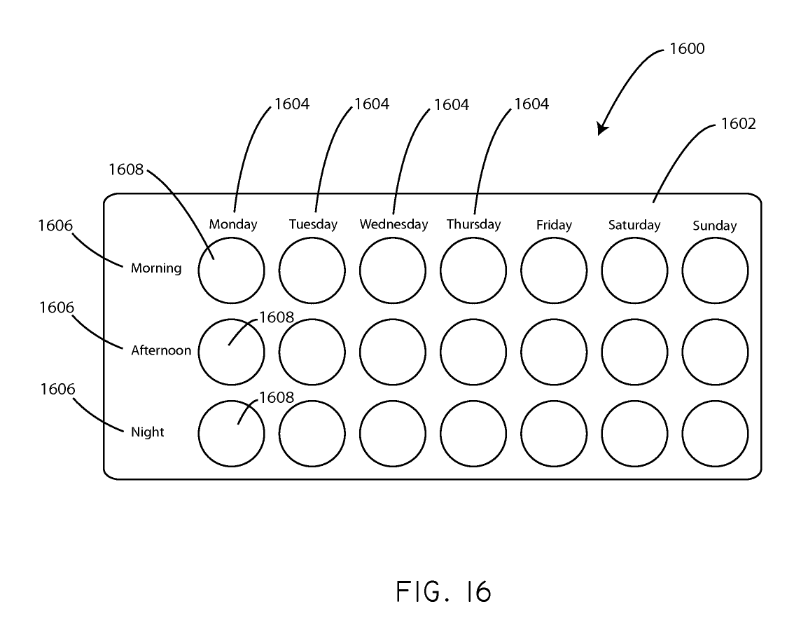

Referring now to FIG. 16, an example of a medical marking tattoo 1600 with indicia is shown. The tattoo 1600 can include a substrate 1602. The substrate 1602 can be formed of various materials, including but not limited to, polymers, cellulosic materials, and the like. In some embodiments, the substrate can also include a layer of a skin compatible adhesive. The tattoo 1600 can include indicia 1604 and 1606. Some of the indicia 1604 can include day information. Some of the indicia 1606 can include time information. The tattoo can also include a plurality of injection target sites 1608.

The method can further include the care giver contacting the carrier sheet with an aqueous solution to cause the tattoo to stick to the patient's skin and peel off the carrier sheet. The method can further include a care giver identifying an appropriate injection target site from amongst the plurality of injection target sites based on the current day and/or time and the day and/or time information provided by the indicia and administering an injection into the identified injection target site. The method can further include a care giver identifying a second appropriate injection target site from amongst the plurality of injection target sites based on the current day and/or time and the day and/or time information provided by the indicia, wherein the second appropriate injection target site is different than the first appropriate injection target site.

In some embodiments, an injection kit is included. The injection kit can include one or more of a plurality of disposable syringes, a plurality of doses of a therapeutic agent (by way of non-limiting example, insulin), a plurality of antiseptic wipes, and one or more medical marking tattoos including those such as described herein. The injection kit can include one or more of a plurality of disposable syringes, a plurality of doses of a therapeutic agent (by way of non-limiting example, insulin), a plurality of antiseptic wipes, and one or marking devices including those such as described herein. In some embodiments, the kits can also include indicator bands. In some embodiments, the syringes can be color coded to match with other elements of the kit such as the medical marking tattoos, the ink of the marking devices and/or the marking devices themselves, and/or the indicator bands. In some embodiments, the kit can also include an instructional video.

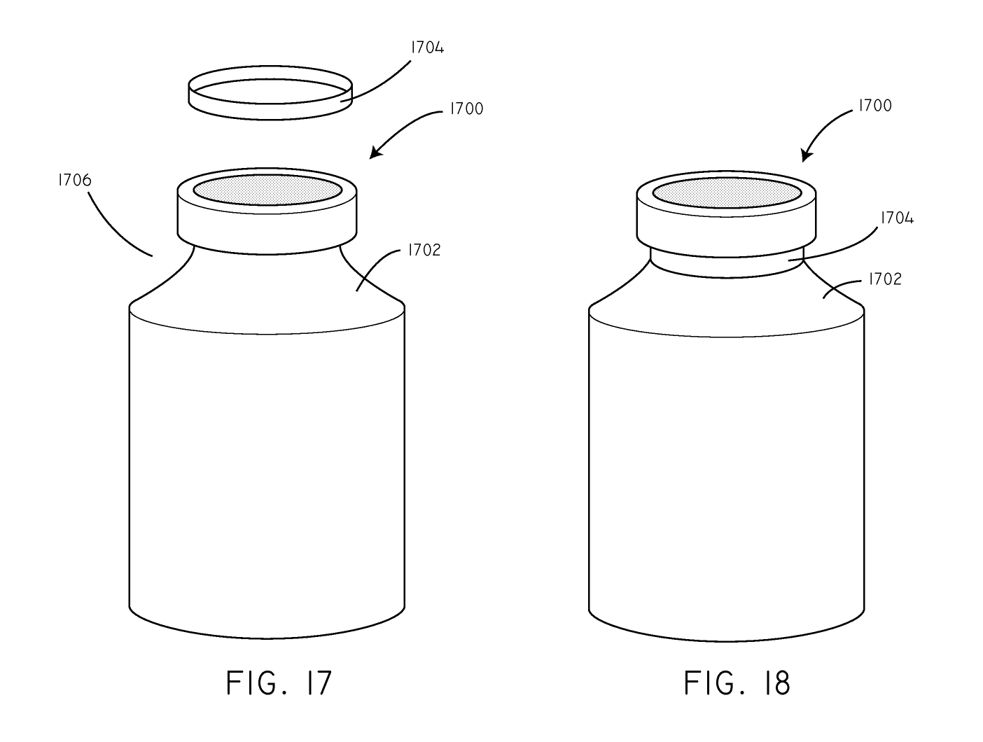

Referring now to FIG. 17, some elements of a kit 1700 are shown in accordance with various embodiments. The kit 1700 can include an indicator band 1704 (or band). The indicator band 1704 can be elastomeric in some embodiments. In some embodiments, the indicator band 1704 can be formed of a polymer. The indicator band 1704 can be configured to fit around a segment of a drug vial 1702 or container. By way of example, the indicator band 1704 can be configured to fit around the neck 1706 of a drug vial 1702 or container. The indicator band 1704 can be of a color that is indicative of the type of drug contained with the drug vial 1702. The indicator band 1704 can be of a color that substantially matches the color of the ink of an injection site marking device. By way of example, an embodiment herein can include an injection site marking device and an indicator band wherein the injection site marking device includes an ink that is substantially the same color as the indicator band. Referring now to FIG. 18, the indicator band 1704 is shown as disposed around the neck of the drug vial 1702.

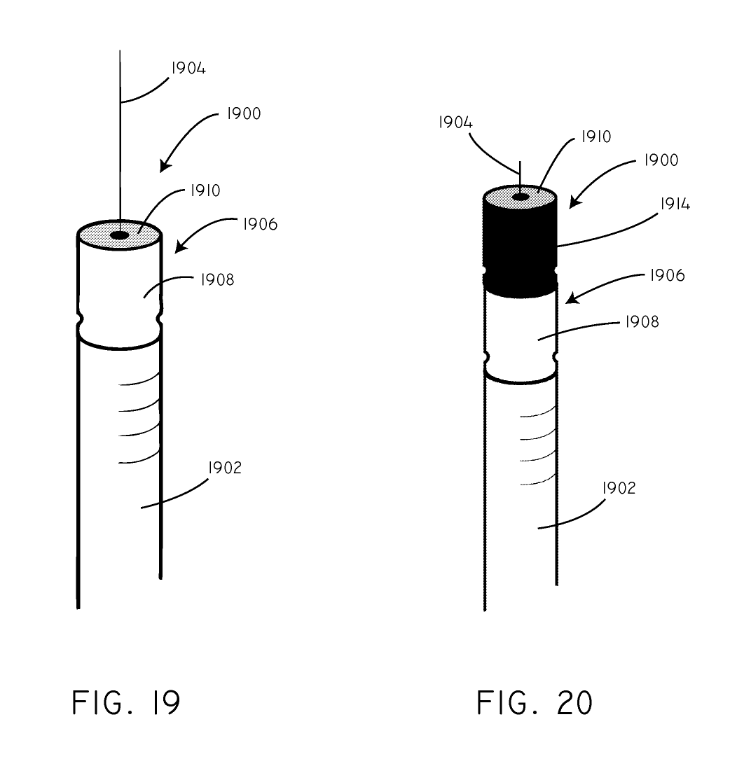

Referring now to FIG. 19, an embodiment of an assembly 1900 is shown including a syringe 1902 including a needle 1904 connected to an injection site marking device 1906. The injection site marking device 1906 can include a platform 1908 that is configured to connect to the syringe 1902. The platform 1908 can connect to the syringe 1902 by way of a press fit mechanism, threads, a snap fit mechanism, luer lock, or the like. Ink retaining media 1910 can be disposed in a layer on top of the platform 1908.

Referring now to FIG. 20, an embodiment of an assembly 1900 is shown including a syringe 1902 including a needle 1904 connected to an injection site marking device 1906. The injection site marking device 1906 can include a platform 1908 that is configured to connect to the syringe 1902. The platform 1908 can connect to the syringe 1902 by way of a press fit mechanism, threads, a snap fit mechanism, luer lock, or the like. The injection site marking device 1906 can also include a needle base 1914. Ink retaining media 1910 can be disposed in a layer on top of the needle base 1914.

In some embodiments, the ink retaining media 1910 can include surface features such that a pattern and/or indicia are deposited as a result of marking the patient. Referring now to

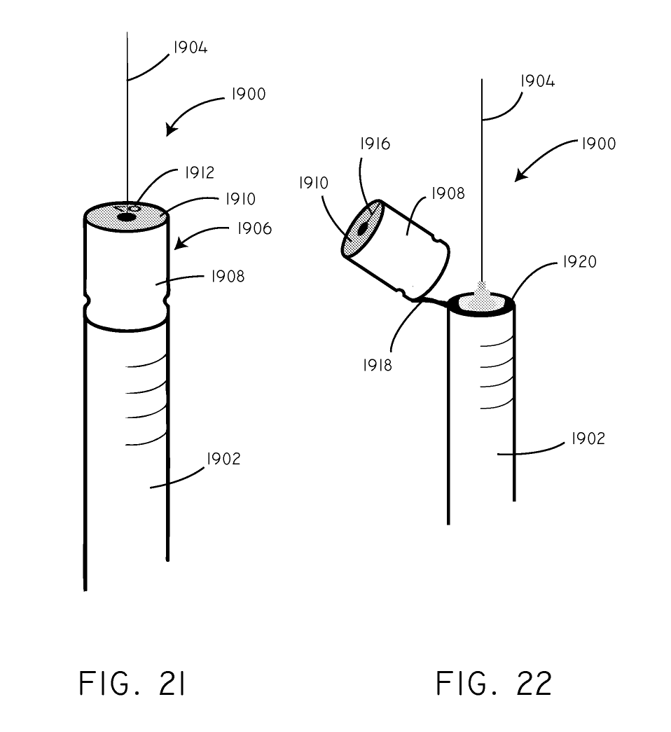

FIG. 21, an embodiment of an assembly 1900 is shown including a syringe 1902 including a needle 1904 connected to an injection site marking device 1906. The injection site marking device 1906 can include a platform 1908 that is configured to connect to the syringe 1902. The platform 1908 can connect to the syringe 1902 by way of a press fit mechanism, threads, a snap fit mechanism, luer lock, or the like. Ink retaining media 1910 can be disposed in a layer on top of the platform 1908. The ink retaining media 1910 can include substantially flat surface with surface features 1912 that are raised above the surface of the surrounding ink retaining media 1910. Alternatively, the ink retaining media 1910 can include a flat surface with surface features 1912 that are disposed below the surface of the surrounding ink retaining media 1910. In this way, the ink retaining media 1910 can be used to imprint indicia (corresponding to either raised portions or lower portions of the ink retaining media) on a patient's skin when the ink retaining media 1910 is contacted therewith. Indicia can include, but are not limited to, those indicative of a day, portion of day, date, time, color, medication, drug lot, caregiver identification, or patient identification.

Referring now to FIG. 22, an embodiment of an assembly 1900 is shown including a syringe 1902 including a needle 1904 connected to an injection site marking device 1906. The injection site marking device 1906 can include a platform 1908 that is configured to connect to the syringe 1902. The platform 1908 can be connected to a hinge 1918 and a retaining ring 1920. The platform 1908 can be moved into position around the needle 1904 via a channel 1916 in the platform through which the needle 1904 can pass. Ink retaining media 1910 can be disposed in a layer on top of the platform 1908.

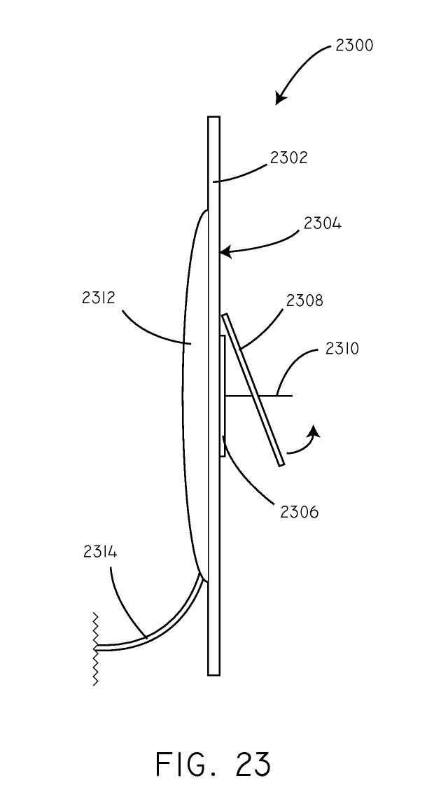

Referring now to FIG. 23, a schematic view of an infusion set marking device 2300 is shown in accordance with various embodiments herein. The infusion set marking device 2300 can include a substrate layer 2302 having a skin contact surface 2304. A layer of adhesive can be disposed over the skin contact surface 2304. A release layer (not shown) can be disposed over the layer of adhesive to protect it until the time for use. The infusion set marking device 2300 can further include ink retaining media 2306 and/or a layer of ink. A cover layer 2308 can also be include that can fit over the ink retaining media 2306 and can be peeled off before use. The infusion set marking device 2300 can further include a needle (or cannula) 2310 and a connection assembly 2312. The connection assembly 2312 can be in fluid communication with a drug supply conduit 2314. The connection assembly 2312 can provide fluid communication between the needle 2310 and the drug supply conduit 2314. The infusion set marking device 2300 can also include various other components such as those described with respect to infusion sets in U.S. Pat. Nos. 8,469,929; 8,287,516 and 7,879,010, the contents of which are herein incorporated by reference.

Various modifications and alterations of this invention will become apparent to those skilled in the art without departing from the scope and spirit of this disclosure, and it should be understood that this invention is not to be unduly limited to the illustrative embodiments set forth herein.

1. (canceled)

2. The injection kit of claim 37, wherein said medical marking tattoo comprises between 1 and 200 injection target sites.

3. The injection kit of claim 37, wherein said medical marking tattoo comprises a temporary skin tattoo comprising several layers of materials interspersed with dye or ink-impermeable layers.

4. The injection kit of claim 37, wherein the medical marking tattoo comprises a glow-in-the-dark material.

5. The injection kit of claim 37, wherein the medical marking tattoo comprises a surface topology characterized by a plurality of raised portions.

6. The injection kit of claim 37, wherein said therapeutic agent medical substance is a substance used to treat diabetes, multiple sclerosis, an autoimmune disease, cancer, or a growth disorder.

7. The injection kit of claim 37, wherein said therapeutic agent medical substance is an injectable medication.

8. The injection kit of claim 37, wherein said therapeutic agent medical substance is insulin, interferon beta-1a or interferon beta-1b.

9-17. (canceled)

18. A method of preventing infection comprising:
applying a medical marking tattoo containing at least one injection target site to a patient's skin where an injection is to be made;
injecting a medical substance into said injection target site contained within said medical marking tattoo using an injection device, wherein the space between the injection target site and previous injection target sites is indicated by the medical marking tattoo;
wherein the space between the injection target site and previous injection target sites as indicated by the medical marking tattoo is at least about 0.5 inches.

19-22. (canceled)

23. An injection site marking device comprising:
a marker, the marker comprising
a syringe channel, the syringe channel configured to fit a syringe head; the syringe channel extending over the length of the marker;
ink retaining media disposed on an end of the marker;
ink disposed within the ink retaining media.

24. The injection site marking device of claim 23, further comprising a removable cap configured to engage the marker.

25. The injection site marking device of claim 23, the injection site marking device comprising a foil liner.

26. The injection site marking device of claim 23, wherein the ink retaining media is ring shaped.

27. The injection site marking device of claim 23, the ink retaining media comprising an aperture.

28. The injection site marking device of claim 23, the marker comprising a recessed portion configured to receive the ink retaining media.

29. The injection site marking device of claim 23, the ink retaining media comprising a substantially flat surface with surface features corresponding to indicia.

30. The injection site marking device of claim 29, wherein the indicia are indicative of a piece of information selected from the group consisting of a day, a portion of day, a date, a time, a color, a medication, a drug lot, caregiver identification, and patient identification.

31. The injection site marking device of claim 23, further comprising an antibacterial compound, an antimicrobial compound, or a tissue preserving medication disposed within the ink retaining media.

32. A method of educating a newly diagnosed patient on proper injection site management comprising:
selecting a patient that is newly diagnosed with a condition requiring therapy that includes injection of a therapeutic agent;
providing the selected patient with a medical marking tattoo;
holding a carrier sheet with the tattoo disposed thereon against the skin of the patient; and
contacting the carrier sheet with an aqueous solution to cause the tattoo to stick to the patient's skin and peel off the carrier sheet.

33-35. (canceled)

36. A method of communicating information amongst caregivers as to the location of an injection:
applying a carrier sheet with a medical marking tattoo disposed thereon against the skin of a patient;
the tattoo comprising multiple injection target sites, distinguished from one another by indicia on the tattoo that includes information selected from the group consisting of day information and time information;
contacting the carrier sheet with an aqueous solution to cause the tattoo to stick to the patient's skin and peel off the carrier sheet;
identifying an appropriate injection target site from amongst the plurality of injection target sites based on the current day and/or time and the day and/or time information provided by the indicia; and
administering an injection into the identified injection target site.

37. An injection kit comprising:
a plurality of disposable syringes;
a plurality of doses of a therapeutic agent;
a plurality of antiseptic wipes, and
one or more medical marking tattoos.

38-40. (canceled)
